(19) United States
(12) Patent Application Publication
(10) Pub. No.: US 2013/0303782 A1
(43) Pub. Date: Nov. 14, 2013

(54) PROCESS FOR PREPARATION OF ALBENDAZOLE

(71) Applicants: Ramkrishna Appaji Rane, Thane (IN); Sushil Naithani, Thane (IN); Rajendra Devendra Natikar, Thane (IN); Sudhakar Verma, Thane (IN); Thangavel Arulmoli, Thane (IN)

(72) Inventors: Ramkrishna Appaji Rane, Thane (IN); Sushil Naithani, Thane (IN); Rajendra Devendra Natikar, Thane (IN); Sudhakar Verma, Thane (IN); Thangavel Arulmoli, Thane (IN)

(73) Assignee: SEQUENT SCIENTIFIC LIMITED, Mangalore, Karnataka (IN)

(21) Appl. No.: 13/989,497

(22) PCT Filed: Nov. 23, 2011
(86) PCT No.: PCT/IN2011/000811

§ 371 Date: Jul. 31, 2013

(30) Foreign Application Priority Data
Nov. 24, 2010 (IN) 3539/CHE/2010

(51) Int. Cl. C07D 235/32

(52) U.S. Cl. CPC C07D 235/32
USPC 548/309.1

(57) ABSTRACT

The present invention discloses a novel, cost-effective process for preparation of a benzimidazole carbamates compound. Specifically, it relates to the process for the preparation of anti-parasite bulk drug albendazole. The process comprises a) thiocyanating 2-nitroaniline of formula VI with ammonium thiocyanated in presence of a halogen to obtain 2-nitro-4-thiocyanoaniline of formula V; b)propylating 2-nitro-4-thiocyanoaniline of formula V with propylbromide in presence of n-propanol and a base in absence of a phase transfer catalyst to obtain 4-propylthio-2-nitroaniline of formula III; C) reducing the nitro group of 4-propylthio-2-nitroaniline prepared in step b) by reacting an aqueous alkali metal sulphide or an alkaline metal sulphide to obtain 4-propylthio-o-phenylenediamine of formula II; and d)condensing 4-propylthio-o-phenylenediamine of formula II with alkali or alkaline earth metal salt of methylcyano carbamate in presence of an acid to form Albendazole of formula I.

### FIELD OF INVENTION

The present invention relates to a novel, cost-effective process for preparation of a benzimidazole carbamates compound. Specifically, it relates to the process for the preparation of anti parasite bulk drug albendazole

### BACKGROUND OF THE INVENTION

Albendazole having chemical name methyl-[6-(propylthio)-1H-benzoimidazol-2-yl]carbamate of formula I, is a member of the benzimidazole compounds used as a drug indicated for the treatment of a variety of worm infestations. Albendazole was first discovered at the SmithKline Animal Health Laboratories in 1972. It is a broad spectrum anthelmintic, effective against roundworms, tapeworms, and flukes of domestic animals and humans. It is efficient antiparasitic agent that has good result of treatment not only to pinworm, ascarid, hookworm and whipworm in the animal bodies such as pig, ox, sheep, but it is also suitable for the treatment to prop up testis trematode, cestode, Echinococcus hydatid cyst, trichina, cysticercus worm etc.

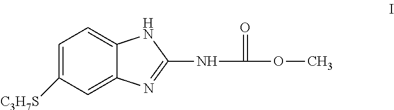

There are number of literatures available which describe the process for preparation of albendazole. U.S. Pat. No. 4,152,522 describes the process in which 2-nitroaniline is thiocyanated to obtain 2-nitro-4-thiocyanoaniline, then alkylated with with n-propylbromide in presence of n-propanol and methyl tributyl ammonium chloride or the tetrabutyl ammonium bromide as the phase-transfer catalyst and an alkali metal cyanide or alkaline metal cyanide to generate 4-propylthio-2-nitroaniline. 4-Propylthio-2-nitroaniline is reduced by sodium sulphide monohydrate in presence of water to obtain 4-propylthio-o-phenylenediamine. This diamine is further reacted with sodium salt of methyl-N-cyano carbamate to obtain the albendazole. In this process phase transfer catalyst as well as an alkali metal cyanide or alkaline metal cyanide is used for condensation of 2-nitro-4-thiocyanoaniline with n-propylebromide, which adds to the cost of production, increases the organic material content in effluent and may facilitate the formation of impurity and uses toxic cyanide compound. The reduction of 4-propylthio-2-nitroaniline is done in presence of water as a solvent which makes the reaction sluggish.

Thus it is highly desirable to develop a process which overcomes most of the drawbacks of the prior art. The present inventors have developed a very cost effective and environment friendly process, which overcomes most of the above stated drawbacks.

### SUMMARY OF THE INVENTION

The principal aspect of the present invention is to provide a process for the preparation of Albendazole comprising:
a) thiocyanating 2-nitroaniline of formula VI with ammonium thiocyanated in presence of a halogen to obtain 2-nitro-4-thiocyanoaniline of formula V;
b) alkylating 2-nitro-4-thiocyanoaniline of formula V with n-propylbromide in presence of an alcoholic solvent and a base in absence of a phase transfer catalyst to obtain 4-propylthio-2-nitroaniline of formula III;
c) reducing the nitro group of 4-propylthio-2-nitroaniline prepared in step b) by reacting with an aqueous alkali metal sulphide or an alkaline metal sulphide or reducing in presence of a metal catalyst in presence of hydrogen to obtain 4-propylthio-o-phenylenediamine of formula II; and
d) condensing 4-propylthio-o-phenylenediamine of formula II with alkali or alkaline earth metal salt of methylcyano carbamate in presence of an acid to form Albendazole of formula I.

The process of the present invention is illustrated in scheme 1 below:

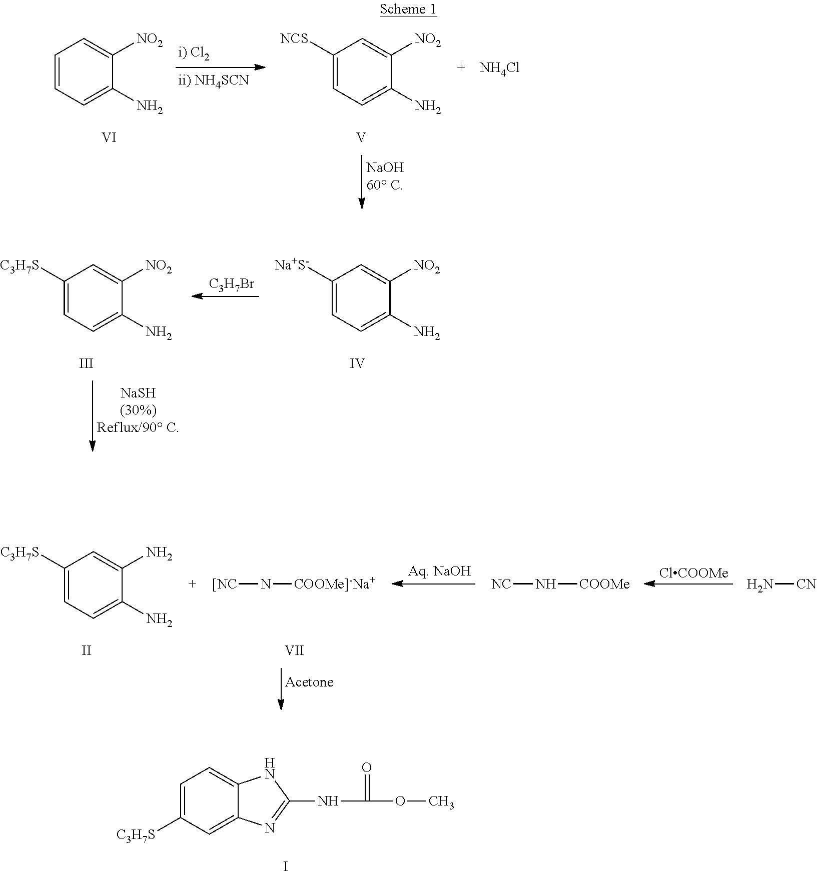

### DETAIL DESCRIPTION OF THE INVENTION

Accordingly in an embodiment of the invention, the thiocyanation of 2-nitroaniline of formula VI with ammonium thiocyanate is carried out in presence of a halogen selected from chlorine and bromine in an alcoholic solvent preferably methanol to obtain 2-nitro-4-thiocyanoaniline of formula V. The reaction is preferably carried out in the temperature range of 0 to 15° C., more preferably in the temperature range of 5 to 10° C.

In another embodiment of the invention, the alkylation in step b) is carried out in an alcoholic solvent selected from methanol, ethanol or n-propanol, preferably n-propanol and a base selected from sodium hydroxide or potassium hydroxide, preferably sodium hydroxide.

In another embodiment of the invention, the reduction in step c) is carried out in an alcoholic solvent like methanol, ethanol, isopropanol, preferably in presence of methanol using aqueous alkali metal sulphide, alkali metal bisulfide or an alkaline metal sulphide selected from sodium hydrogen sulphide and sodium disulfide, preferably sodium hydrogen sulfide. The reduction may be carried out in presence of a metal catalyst such as Raney nickel at a hydrogen pressure of 8 to 12 kg/cm² preferably at 10 kg/cm² for 3 to 7 hours preferably for 4-6 hours. The obtained 4-propylthio-o-phenylenediamine of formula II is distilled at preferably less than 185° C. under high vacuum at 1 mm/Hg.

In yet another embodiment of the invention, the condensation of 4-propylthio-o-phenylenediamine of formula II with alkali or alkaline earth metal salt of methylcyano carbamate is carried out in presence of acetone and water as a solvent and a mineral acid preferably concentrated hydrochloric acid at a pH in the range 4 to 4.5. The alkali metal salt of methylcyano carbamate is preferably sodium methylcyano carbamate.

The present invention is advantageous over prior art, some of them are stated below:
1. The present invention avoids the use of phase transfer catalyst as well as an alkali metal cyanide or alkaline metal cyanide to generate 4-propylthio-2-nitroaniline. This minimizes the organic material content in effluent, and reduce the production cost significantly.
2. The present invention uses methanol as solvent for the reduction which reduces the sluggishness and makes the reaction very smooth and fast. The catalytic reduction in presence of a metal catalyst is a green reaction and environment friendly.
3. The distillation of diamine is carried out in Agitated Thin Film Evaporator (ATFE) to remove low boiling and high boiling impurities at 170° C. to 185° C. under high vacuum at 1 mm/Hg. The contact time of diamine in ATFE is very less hence the decomposition of diamine is minimised resulting increase in yield and purity.
   The present invention can be illustrated by the following examples, which are not to limit the scope of invention.

### EXAMPLE 1

### Preparation of Methyl-[6-(Propylthio)-1H-Benzoimidazol-2-yl]carbamate (I)

(a) Preparation of 2-Nitro-p-Thiocyanoaniline

2-Nitroaniline (360 kg) was treated with ammonium thiocyanate (407 kg) in methanol at room temperature. The reaction mixture was stirred and cooled to below 10° C. Chlorine gas was purged for 6 hrs and maintained for 1 hr. After completion of the reaction, water was added and stirred for 1 hr at 20° C. The reaction mass was filtered, washed with water, and dried at 80° C.

Weight: 504 kg.

(b) Preparation of 2-Nitro-p-Thiopropyl-Aniline

A suspension of 2-Nitro-p-thiocyanoaniline (800 kg), water and n-propanol (2000 L) was made and caustic lye (700 kg) was added slowly to it below 35° C. The reaction mass was heated to 40° C., and n-propylbromide was added to it and further heated to 60° C. After completion of the reaction, the reaction mass was subjected to distillation and even traces of n-propanol was distilled out under vacuum. The lower aqueous layer was separated out and charged sodium chloride (17 kg) in water (800 L) to the above organic layer and heated to 90° C., maintained for 1 hr to separate and obtain the liquid title product.

Yield:800 kg (92.6%).

(c) Preparation of 4-Propylthio-o-Phenylenediamine

Sodium hudrogensulfide (3200 L) was added slowly to a mixture of liquid 2-nitro-p-thiopropyl-aniline (800 kg) and methanol(1600 L) at 50° C. and heated to reflux at 65-70° c. After completion of the reaction, methanol was distilled out completely and the layers were separated. The upper organic layer was subjected to a high vacuum distillation to obtain 550 kg of title compound.

(d) Preparation of 4-Propylthio-o-Phenylenediamine

2-Nitro-p-thiopropyl-aniline was reduced in Methanol 5200 L by loading 20 kg Raney nickel at 100° C. with 10 kg/cm2 pressure for 4-6 hrs followed by isolation of spent Raney nickel. Crude diamine oil was isolated by complete removal of methanol. Crude diamine oil was further distilled under high vacuum to obtain 550 kg of title compound.

(e) Preparation of Methyl-N-Cyano Carbamate Sodium Salt

In cyanamide (242 kg) and water (800 L) at below 20° C., methylchloroformate (300 kg) and caustic lye (280 kg) were added simultaneously while maintaining the temperature below 10° C. and pH 7-7.5 was maintained. After addition, the pH was adjusted to 8-8.5 using caustic lye, and then maintained for 2 hrs at 10° C. to obtain the title compound.

(f) Preparation of Albendazole

4-Propylthio-o-phenylenediamine (400 kg) was treated with acetone (400 L). Then water (380 L) and conc. HCl (360 kg) was added to it. Exothermic reaction observed upto 48° C. Reaction mass was cooled to room temperature and methyl-N-Cyano Carbamate was added. The reaction mass was heated to 80-85° C. The pH was adjusted to 4-4.5 by concentrated HCl and centrifuged. The material and washed with hot water, tap water, methanol and finally with acetone.

Weight: 500-520 kg.

1. A process for the preparation of Albendazole of formula I comprising: 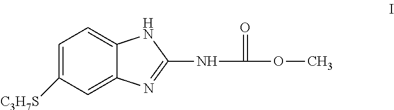
a) thiocyanating 2-nitroaniline of formula VI with ammonium thiocyanate in the presence of a halogen to obtain 2-nitro-4-thiocyanoaniline of formula V; 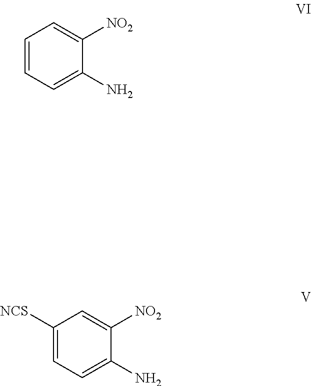
b) alkylating 2-nitro-4-thiocyanoaniline of formula V with n-propylbromide in presence of an alcoholic solvent and a base in the absence of a phase transfer catalyst to obtain 4-propylthio-2-nitroaniline of formula III: 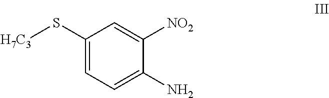
c) reducing the nitro group of 4-propylthio-2-nitroaniline to obtain 4-propylthio-o-phenylenediamine of formula II; and 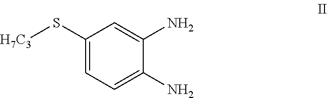
d) condensing 4-propylthio-o-phenylenediamine of formula II with an alkali or alkaline earth metal salt of methylcyano carbamate in presence of an acid to form Albendazole of formula I.

2. A process according to claim 1, wherein the halogen in step (a) is chlorine or bromine.

3. A process according to claim 1, wherein the alcoholic solvent in step (b) is selected from the group consisting of methanol, ethanol and n-propanol.

4. (canceled)

5. A process according to claim 1, wherein the nitro group of 4-propylthio-2-nitroaniline is reduced in the presence of Raney nickel at a hydrogen pressure of 10 kg/cm² for 4 to 6 hrs to obtain 4-propylthio-o-phenylenediamine of formula II.

6. A process according to claim 1, wherein the alkali metal salt of methylcyano carbamate is sodium methylcyano carbamate

7. A process according to claim 1, wherein the condensation of 4-propylthio-o-phenylenediamine of formula II with the alkali or alkaline earth metal salt of methylcyano carbamate is carried out in the presence of acetone and water as a solvent and in the presence of a mineral acid at a pH in the range of 4 to 4.5.

8. A process according to claim 1, wherein the reducing step comprises reducing the nitro group of 4-propylthio-2-nitroaniline by catalytic hydrogenation.

9. A process according to claim 1, wherein the reducing step comprises reducing the nitro group of 4-propylthio-2-nitroaniline by reaction with a sulfide salt.

10. A process according to claim 1, wherein the reducing step comprises reducing the nitro group of 4-propylthio-2-nitroaniline by reaction with a sulfide salt selected from the group consisting of alkali metal sulfides, alkali metal bisulfides, and alkaline metal sulphides.

11. A process according to claim 1, wherein the reducing step comprises reducing the nitro group of 4-propylthio-2-nitroaniline by reaction with a sulfide salt selected from the group consisting of sodium hydrogen sulphide and sodium disulfide.

12. A process according to claim 1, wherein the base in step (b) is selected from the group consisting of sodium hydroxide and potassium hydroxide.
